(19) United States
(12) Patent Application Publication
(10) Pub. No.: US 2023/0287369 A1
(43) Pub. Date: Sep. 14, 2023

(54) A NEW, NON-SPECIFIC THERMOLABILE NUCLEASE ACTIVE AT LOW TEMPERATURE, IN WIDE PH RANGE AND HIGH CONCENTRATION IN SALTS

(71) Applicant: Blirt S.A., Gdansk (PL)

(72) Inventors: Marcin Olszewski, Gdansk (PL); Rafat Walkusz, Gdynia (PL); Wioleta Lademan, Banino (PL); Jan Balsewicz, Gdynia (PL); Arkadiusz Popinigis, Gdansk (PL); Dominik Zietkowski, Gdynia (PL); Krzysztof Kur, Reda (PL); Robert Brodzik, Gdansk (PL)

(73) Assignee: Blirt S.A., Gdansk (PL)

(21) Appl. No.: 17/642,697

(22) PCT Filed: Sep. 14, 2020
(86) PCT No.: PCT/PL2020/000076

§ 371 Date: Mar. 13, 2022

(30) Foreign Application Priority Data
Sep. 13, 2019 (PL) P.431144

(51) Int. Cl. C12N 9/22

(52) U.S. Cl. CPC C12N 9/22

(57) ABSTRACT

The subject matter of this invention is a new thermolabile PPR nuclease or its enzymatically active fragment exhibiting high catalyst activity in difficult reaction conditions (particularly high concentrations of salts and other additives commonly used in the processes of the purification of proteins and viruses, low temperatures and wide range of pH), where the nuclease sequence is SEQ. 2 or a sequence that shares at least 40% of its identity. The subject matter of the invention is also the gene encoding PPR nuclease or its enzymatically active fragment; a particle of nucleic acid encoding the PPR nuclease or its enzymatically active fragment; expression plasmid including the sequence of the PPR encoding gene; recombinant strains of Escherichia coli JM109(DE3) pD454-PPR-AmpR and E. coli ArcticExpress (DE3) pD454-PPR-AmpR; the method of PPR nuclease protein production, application of PPR nuclease in processes of purification of recombinant proteins of significantly lower DNA content. As well as for decontamination of reactive reagents and mixtures for PCR, qPCR, RT-PCR, RT-qPCR, RCA, LAMP and NGS in order to obtain higher sensitivity and specificity of relevant genetic analyses; application of the PPR nuclease in processes of virus vector purification (particularly lentiviruses [LV], adenoviruses [AV, AAV] and retroviruses [RV] used in modern gene and cell therapies (chimeric antigen receptor [CAR] T-cell immunotherapy)); application of the PPR nuclease in exosome purification processes for therapeutic or diagnostic purposes; application of the PPR nuclease in processes of recombinant protein purification, particularly enzymes, antibodies, vaccination antigens, products used in cell therapies and other therapeutic proteins.

### SEQUENCE LISTING

The instant application contains a Sequence Listing which has been submitted electronically in ASCII format via EFS-Web and hereby incorporated by reference in its entirety. Said ASCII copy, created on 6 Sep. 2022, is named BLI001US1_ST25_06Sept2022.txt and is 12,288 bytes in size.

The subject matter of the invention is a new, thermolabile, non-specific PPR nuclease or its enzymatically active fragment, or a sequence that shares at least 40% of its identity, active at low temperature, in wide pH range and high concentrations of salts (e.g. NaCl, KCl, MgCU, MgSCE, (NH\hat{ }iSCE). The subject matter of the invention is also a gene-encoding PPR nuclease or its enzymatically active fragment; a particle of nucleic acid-encoding PPR nuclease or its enzymatically active fragment; expression plasmid including the sequence of the PPR-encoding gene; a recombinant strain of Escherichia coli JM109(DE3) pD454-PPR-AmpR and a strain of Escherichia coli ArcticExpress (DE3) pD454-PPR-AmpR; the method of PPR nuclease protein production, application of the PPR nuclease in processes of purification of recombinant proteins of significantly lower DNA content as well as for decontamination of reagents and mixtures for PCR, qPCR, RT-PCR, RT-qPCR and NGS in order to obtain higher sensitivity and specificity of relevant genetic analyses; application of the PPR nuclease in the processes of virus vector purification (particularly lentiviruses [LV], adenoviruses [AV, AAV] and retroviruses [RV] used in modern gene and cell therapies (chimeric antigen receptor [CAR] T-cell immunotherapy); application of the PPR nuclease in exosome purification processes for therapeutic or diagnostic purposes; application of the PPR nuclease in processes of recombinant protein purification, particularly enzymes, antibodies, vaccination antigens, products used in cell therapies, and other therapeutic proteins.

Currently, the most popular non-specific nuclease is Benzonase® (Merck, USA), whose optimal temperature of activity is 37° C., and the main disadvantages include no possibility of effective inactivation by high temperature and limited tolerance to increased concentration of salts. Similar parameters are exhibited by Benzonase®-related products of generic character, such as e.g. Denarase, produced in another host-Bacillus sp. (c-LEcta, Germany). Another example of an enzyme of similar characteristics is Cyanase™ nuclease, derived from another microorganism (RiboSolutions, USA). However, the main assumption of inventors is to obtain a non-specific nuclease derived from psychro- and halophile microorganisms, retaining significant activity below 20° C. and even in refrigerated conditions (4-8° C.), maintaining optimal activities in a wide spectrum of salt concentrations and pH, and characterized by a lower temperature of irreversible enzyme inactivation. Currently, on world markets there are only two non-specific nucleases exhibiting significant activity at lower temperatures. These are Cryonase™ (Takara, Japan), derived from a psychotropic organism and HL-SAN (ArcticZymes, Norway). However, compared to the subject invention, these enzymes are characterised by a lower tolerance to high concentrations of salts, weaker activity at low temperatures (<20° C.) and a narrower pH tolerance range (residual activity at pH<7.0).

DNA contamination, commonly occurring in protein products produced in microorganisms, poses a significant problem during industrial manufacturing of recombinant proteins and enzymes, especially for diagnostic, therapeutic and scientific purposes.

Enzymes of a significantly lower nucleic acid contamination (so called “DNA-free”) are ideal for precise diagnostics based on amplification and/or DNA ligation (among others: PCR, qPCR, RT-qPCR, NGS, RCA, LAMP), where the highest sensitivity, specificity and the lack of ambiguous or false positive results are required. Even traces of foreign DNA may lead to obtaining artefacts in the above-mentioned, supersensitive techniques. The problem of DNA contamination escalates when detected DNA is in a low quantity. The signal from contaminated DNA may interfere with low-copy DNA detection, being the subject of the measurement, significantly affecting the sensitivity and reliability of the test.

Commercial suppliers of enzymes and reagents (among others DNA polymerases, PCR master mixes, reagents for NGS) confirm the significance of nucleic acid contamination, and offer DNA-free products, which differ from conventional reagents in terms of their production technology and quality control. However, the level of contamination of these products is very often far from expectations as it strongly relies on the sensitivity of the DNA contamination detection method (according to literature findings, the majority of companies offer DNA-free polymerases, containing from 10 to 1000 copies of the DNA genome per 1 U of enzyme).

The production of therapeutic proteins and active substances for medicinal products, due to high quality standards, also requires the removal of process contaminants, particularly related to DNA (host and exogenous). The amount of the remaining DNA must usually be limited to 100 pg per drug dosage (e.g. in the case of therapeutic antibodies) and for some vaccines to 10 ng per drug dosage. These values are determined by the guidelines of the World Health Organization (WHO), as well as the Food and Drug Administration (FDA) and the European Medicines Agency (EMEA).

An ideal tool for the purification of contaminating nucleic acids seems to be the application of an appropriate non-specific and universal nuclease, characterized by high activity at low temperatures (4-22° C.), wide range of pH (6.0-10.0), and a high concentration of salts and other additives commonly used in purification processes (downstream processing) which could be inactivated at a temperature that is safe for enzymes and biopharmaceuticals that are being purified (proteins, enzymes, antibodies, antigens, virus vectors for gene therapy etc.).

Particularly in terms of effective purification from nucleic acids of virus vectors (among other lentiviruses (LV), adenoviruses (AV, AAV), retroviruses (RV)), used in modern gene and cell therapies (such as CAR-T therapies), it is highly desirable to apply purification conditions at high salt concentrations (250-1000 mM NaCl) and pH within 6.0-8.0 (Kramberger et al., Hum Vaccin Immunother. 2015; 11(4): 1010-21. doi: 10.1080/21645515.2015.1009817), i.e. the optimal conditions for PPR nuclease action. Such conditions significantly facilitate the digestion of nucleic acids of the host cells composing chromatin, as well as change the viscosity of solutions containing virus vectors or proteins facilitating their purification. Additionally, they are indispensable for effective binding of purified virus vectors or proteins to the stationary phase. This is of key importance for increased efficiency of the production processes and significant reduction in production costs.

In recent years interest regarding enzymes from psychrophilic microorganisms (i.e. ones adapted to living at low temperatures) has been growing. The great significance of these enzymes is related to their high activity at low temperatures (generating production savings) and thermal liability, thanks to which their effective, fast and selective inactivation is possible (after purification process) by a slight temperature increase (that does not damage the product which is under enzymatic treatment).

The subject-matter, a thermolabile, non-specific nuclease, may be applied for the production of enzymes that are free from nucleic acids (e.g. DNA-free polymerases, reverse transcriptases, or ligases). These are very expensive and not widely available enzymes, which are often desirable for specialist technologies of molecular biology and in vitro diagnostics. The PPR nuclease, the subject matter of our invention, may also be used by pharmaceutical and cosmetics companies for the purification of products of natural origin from nucleic acids. For this purpose the pharmaceutical market currently uses mesophilic Benzonase® (Merck), which is characterized by low tolerance to monovalent and bivalent salts in the reaction environment.

An international publication, WO2006095769, has described a polypeptide of endonuclease activity derived from Shewanella sp., psychrophile microorganisms which exhibit high activity at low temperatures. It may remove any nucleic acid present in protein solution as well as lower viscosity of protein extract. However, its inactivation poses certain difficulties as, in accordance with literature reports (Saramiento et ah, Front Bioeng Biotechnol. 2015; 3: 148.), it requires incubation at 70° C. for 30 minutes (at such a high temperature many recombinant proteins may denature).

Moreover, an international publication, WO2013/121228, presents a non specific endonuclease and its enzymatically active fragment, available under the HL-SAN trade name. This invention regards endonucleases, which are inactivated by mild temperature conditions, exhibiting thermolabile properties. This invention also includes the removal of contaminating polynucleotides from biologicals by the application of this endonuclease. This invention also concerns the prevention of false positive results in the amplification reaction of nucleic acid by the application of endonuclease, in particular, in the amplification reaction by the PCR method.

The aim of this invention was to obtain a thermolabile, non-specific nuclease of better properties maintaining high activity at temperatures below 20° C., in particular in refrigerated conditions (4-8° C.), in high concentrations of salts and possibly wide pH range. Moreover, PPR nuclease is compatible with the majority of buffers and additives used in bioprocess. This enzyme, hydrolyzing nucleic acid, may be a highly valuable tool used for the production of recombinant proteins with a lower content of nucleic acids, enzymes, antibodies, vaccination antigens, exosomes, vims vectors for gene or cell therapies, the preparation of products used in cell therapies, and the purification of other therapeutic proteins from DNA and RNA contaminations (e.g. enzymes for molecular biology and precision in vitro diagnostics, proteins and virus vectors for the biopharmaceutical industry, and biological components for the veterinary and cosmetics industries).

The subject matter of the invention is a PPR nuclease or its enzymatically active fragment, where the nuclease sequence is SEQ. ID NO: 2 or the sequence that shares at least 40% of its identity.

The PPR nuclease or its enzymatically active fragment is irreversibly inactivated after incubation for 15 minutes at 52° C. in the presence of 1-5 mM DTT, and it is possible to lower the inactivation temperature by longer incubation with DTT.

The PPR nuclease or its enzymatically active fragment is generally active in concentrations of the following salts: NaCl: 0-1400 mM. MgCh 5-200 mM, urea: 0-6000 mM, ammonium sulphate: 0-200 mM, imidazole: 0-400 mM.

The gene encoding PPR nuclease or its enzymatically active fragment with a sequence is presented as SEQ. ID NO: 1.

The particle of nucleic acid encoding the PPR nuclease or its enzymatically active fragment as defined in Claims 1-3 or encoding the protein containing the above-mentioned PPR nuclease or its enzymatically active fragment.

Expression plasmid, pD454-PPR-AmpR, containing the sequence of the PPR-encoding gene in accordance with Claim 4. In addition, the plasmid includes: a T7 phage promoter or another one active in the E. coli expression system. The plasmid has the SEQ. ID NO: 4 sequence.

Recombinant strains of Escherichia coli JM109(DE3) pD454-PPR-AmpR or Escherichia coli ArcticExpress (DE3) pD454-PPR-AmpR are transformed with the above-described plasmid.

The manner of PPR nuclease protein production, where recombinant strains of Escherichia coli JM109(DE3) pD454-PPR-AmpR or Escherichia coli ArcticExpress (DE3) pD454-PPR-AmpR are cultured on medium and later induction of the PPR nuclease gene expression is performed by adding IPTG; the protein is isolated and purified.

The manner of isolation and purification of the PPR nuclease, or its enzymatically active fragment as defined above, which involves expression of the previously described nuclease or its fragment in a relevant host cell and consequently separation of the nuclease from the host cell and/or medium where the cells were cultured.

PPR nuclease application in the processes of the purification of recombinant proteins of significantly lower DNA content and for decontamination of reagents and reaction mixtures for PCR, qPCR, RT-PCR, RT-qPCR, RCA, LAMP and NGS in order to obtain increased sensitivity and specificity of the relevant genetic analyses.

Application of the PPR nuclease in the processes of virus vector purification (particularly lentiviruses [LV], adenoviruses [AV, AAV] and retroviruses [RV]), used in modern gene and cell therapies (e.g. chimeric antigen receptor [CAR] T-cell immunotherapy).

Application of the PPR nuclease in processes to purify exosomes used for therapeutic and diagnostic purposes.

Application of the PPR nuclease in the processes of recombinant protein purification, particularly of enzymes, antibodies, vaccination antigens, products used in cell therapies and other therapeutic proteins.

Application of the PPR nuclease in the pharmaceutical, veterinary and cosmetics industries.

Application of the PPR nuclease in the pharmaceutical and biotechnology industries to remove DNA contamination in culture media for mammalian fermentation and microbiological processes.

The terminology used above in Description and Patent Claims has the following meaning:
Nuclease—this term refers to the enzyme that hydrolyzes a phosphodiester bond in the polynucleotide chain of nucleic acids (DNA or RNA).
Non-specific nuclease—enzyme hydrolyzing all types of nucleic acids (including ssDNA, dsDNA, circular DNA, ssRNA, dsRNA).
Psychrophilic organism—organism that lives at low temperatures (below 20° C.).
Psychrotropic organism—organism tolerating low temperatures (may live at low temperatures but it is not necessary).
Halophilic organism—organism tolerating high concentration of salts, that lives in saline waters or soils.
PPR nuclease—non-specific nuclease that is the subject matter of the invention of SEQ ID NO: 2.

### DESCRIPTION OF FIGURES AND SEQUENCES

FIG. 1—showing the scheme of the pD454-PPR expression plasmid.

FIG. 2—showing pH influence on PPR nucleolytic activity depending on NaCl salt concentration. Measurement using modificated Kunitz's test, conditions: 20 mM MgCh, temperature of 22° C.

FIG. 3—showing temperature and high salts (500 mM NaCl+100 mM MgCL) influence on PPR nucleolytic activity. Measurement using modificated Kunitz's test.

FIG. 4—showing influence of Mg², ions on PPR nucleolytic activity in selected pH and temperature conditions. Maximum PPR activity is obtained at 50-150 mM concentration at 37° C. in a pH 8.0 buffer. At ambient temperature (22° C.) in a pH 6.5 buffer optimal activity is obtained at significantly lower Mg²⁺ ion concentrations (20-50 mM).

FIG. 5—showing PPR inactivation in various temperature conditions in the presence of 5 mM DTT. Complete PPR inactivation is obtained at 52° C.

FIG. 6—showing the comparison of nucleolytic activity values of PPR, HL-SAN and Benzonase® nucleases in buffers of various NaCl concentrations (0, 250, 500 mM), in pH 7.0, 8.0, 9.0, respectively. The remaining reaction conditions are as follows: temperature 22° C., 50 mM Tris, 20 mM MgCk (5 mM MgCk used for Benzonase®). The greatest competitive advantages PPR shows in high NaCl concentrations (250-500 mM), commonly used in the processes of purification of recombinant proteins and virus vectors. The advantage of PPR over the HL-SAN nuclease, whose characteristics are the most similar, increases in correlation with decreasing pH (7.0-8.0).

FIG. 7—showing the comparison of values of the PPR, HL-SAN and Benzonase® nucleases' nucleolytic activities in DMEM medium, commonly used for mammalian in vitro cell culture, in which recombinant proteins, virus vectors and other biological therapeutics are produced.

FIG. 8—showing the comparison of values of the PPR, HL-SAN and Benzonase® nucleases' nucleolytic activities in buffers with similar physiological salt concentration (PBS and TBS) with the addition of 500 mM NaCl, commonly used in the methodology of recombinant protein purification.

FIG. 9—showing detection of contaminating host DNA (E. coli) using qPCR methods in samples of UDGase (UDG) and purified UDGase using the PPR nuclease (UDG+PPR).

FIG. 10—showing the removal of genomic DNA contamination from post-culture medium of CHO cells producing cetuximab and bevacizumab monoclonal antibodies.

SEQ. ID NO: 1—showing the PPR nuclease nucleotide sequence.

SEQ. ID NO: 2—showing the amino acid sequence of the PPR nuclease protein.

SEQ. ID NO: 3A—showing the amino acid sequence of the PelB signal peptide.

SEQ. ID NO: 3B—showing His6-tag, allowing for purification.

SEQ. ID NO: 4—showing the sequence of the recombinant pD454-PPR-AmpR expression plasmid.

### EXAMPLES

The invention is illustrated by the following examples of its performance, without any limitation of its application.

### Example 1

Obtaining the Expression Plasmid pD454-PPR-AmpR.

To obtain the expression plasmid pD454-PPR-AmpR, purified by ethanol precipitation, the DNA fragment of the SEQ. ID NO: 1 pattern is digested by the Sap1 restriction enzyme, and following that it is ligated with DNA of the pD454-SR plasmid vector (ATUM, Newark, Calif. 94560, USA), digested by the same restriction enzyme.

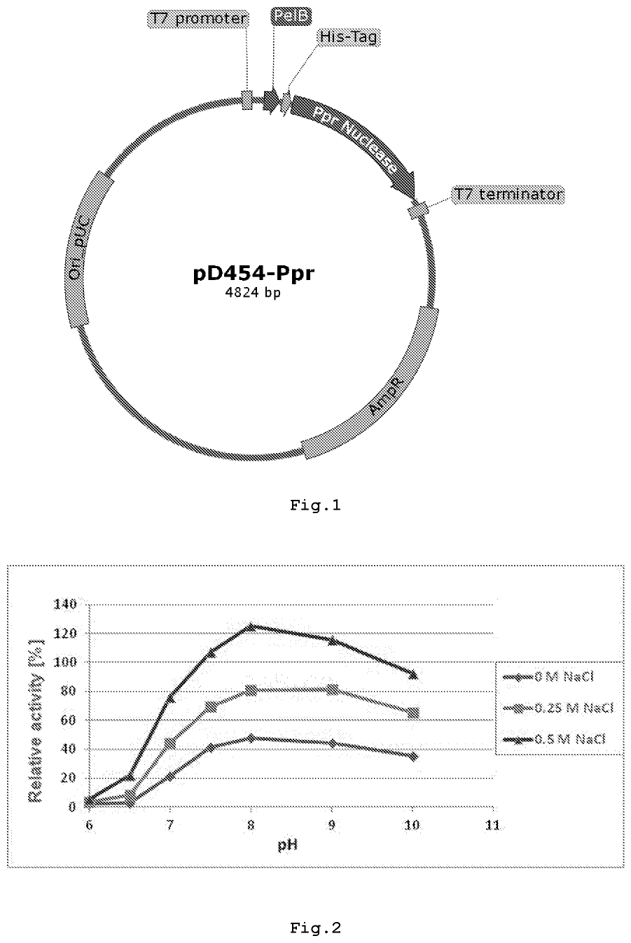

The ligation mixture transforms the competent TOPI OF Escherichia coli cells that are placed onto Petri dishes with LA medium (1% peptone; 0.5% yeast extract; 1% NaCl; 1.5% agar) containing ampicillin 100 pg/ml. As a result of plasmid DNA isolation, the expression plasmid of the pD454-PPR-AmpR and SEQ. ID NO: 4 sequence is obtained from the developed bacterial colonies. The map of the pD454-PPR-AmpR plasmid is shown in FIG. 1.

### Example 2

Obtaining Recombinant Strains of E. coli JM109(DE3) pD454-PPR-AmpR or E. coli ArcticExpress (DE3) pD454-PPR-AmpR.

To obtain recombinant strains of E. coli JM109(DE3) pD454-PPR-AmpR or E. coli ArcticExpress (DE3) pD454-PPR-AmpR transformations of E. coli JM109(DE3) or E. coli ArcticExpress (DE3) cells are performed with circular DNA of pD454-PPR-AmpR expression plasmid obtained as described in Example 1 (SEQ. ID NO: 4). Bacteria cells are placed onto LB medium (1% peptone; 0.5% yeast extract; 1% NaCl) containing ampicillin 100 pg/ml and then the obtained colonies of recombinant strains of E. coli JM109(DE3) pD454-PPR-AmpR or E. coli ArcticExpress (DE3) pD454-PPR-AmpR are used for biosynthesis of the PPR nuclease.

### Example 3

Obtaining the PPR Nuclease Using the Cells of Recombinant Strains of E. coli JM109(DE3) pD454-PPR-AmpR or E. coli ArcticExpress (DE3) pD454-PPR-AmpR.

The recombinant strains of E. coli JM109(DE3) pD454-PPR-AmpR or E. coli ArcticExpress (DE3) pD454-PPR-AmpR obtained in accordance with Example 2 are cultured in LB medium (1% peptone; 0.5% yeast extract; 1% NaCl) containing ampicillin 50 pg/ml at 37° C. for 16-18 h. Following that, the overnight culture is used to inoculate a medium of identical contents, at a 1:50 ratio. The culture is continued at 30° C. until ODeoo (optical density)=0.4-0.5 is obtained, and then induction of PPR nuclease gene expression is performed by adding IPTG to a final concentration of 0.2 mM. The culture is maintained at 18° C. for 20-22 h and then bacteria cells are separated from the medium by centrifugation. Cell sedimentation suspended in the buffer contains 20 mM Tris-HCl (pH 8.0), 500 mM NaCl, 10 mM imidazole, 5 mM MgCb; at least 5 ml of buffer solution per 1 g of cell sedimentation.

Following that, the suspension of cells undergoes disintegration using ultrasounds, performing 3 cycles of sonication; energy intensity 100 J/ml of suspension. The obtained cell lysate is centrifuged at 16000 RCF in order to remove insoluble proteins and cell fragments, and then it is filtrated through a 0.2 mM membrane. The PPR nuclease protein is separated from the remaining bacterial proteins applying the immobilized metal affinity chromatography method (IMAC) using the stationary phase with immobilized bivalent ions of nickel. The PPR nuclease bound to the stationary phase is then washed out by the buffer containing 20 mM Tris-HCl (pH 8.0), 500 mM NaCl, 300 mM imidazole, 5 mM MgCK The fraction containing the PPR nuclease undergoes dialysis with a buffer solution containing 20 mM Tris-HCl (pH 8.0), 500 mM NaCl, 5 mM MgCb; at least 100 ml per 1 ml enzyme, for at least 18 hours in refrigerated conditions. The obtained enzyme formula is mixed 1:1 with glycerol and frozen at −20° C. Measurement of protein concentration is performed using the spectrophotometric method, at 280 nm wavelength.

### Example 4

### Examination of the Enzymatic Properties of the PPR Nuclease Recombinant Protein.

Specific nucleolytic activity regarding nucleic acids necessary to define the optimal conditions of enzyme activity and inactivation was determined for the obtained recombinant PPR nuclease in accordance with Example 3.

In order to determine PPR nuclease nucleolytic activity serial dilutions of the enzyme are incubated in a reaction buffer containing 20 mM Tris-HCl (pH 8.0), 20 mM MgCb and 1 pg pUC19 plasmid DNA of 20 m [symbol] volume for 10 min. The reaction is stopped by adding 5 m [symbol] 25 mM DTT solution to a final concentration of 5 mM and the sample is heated to 55° C. for 10 min. As a control, 1 pg of pUC19 plasmid DNA is incubated without the addition of nuclease. Subsequently, the samples are loaded onto a 1% agarose gel and DNA separation is performed in gel at 130 voltage for 40 min. The remaining, nondegraded DNA in gel is stained with ethidium bromide and documented by taking a photography of the gel. 1 U activity is determined as the enzyme quantity essential for total degradation of 1 pg of pUC19 plasmid DNA in 10 min. at 37° C.

In order to determine PPR nuclease specific activity serial dilutions of the enzyme are incubated in a reaction buffer containing 20 mM Tris-HCl (pH 8.0), 20 mM MgCh and 100 pg of herring sperm genomic DNA of 300 m [symbol] volume at 37° C. for 30 min. The reaction is stopped by adding 300 m [symbol] 4% solution of perchloric acid to 2% final concentration and sample incubation on ice for 60 min. As a control, 100 pg DNA is incubated without the addition of nuclease. Afterwards, the samples are centrifuged for 10 minutes until separation of the precipitate of non-degraded DNA. The content of free nucleotides and oligonucleotide fragments smaller than 10 bp is determined in supernatant by measuring absorbance at 260 nm wavelength. 1 U nuclease activity is determined as enzyme quantity causing increase of absorbance in the studied sample at 260 nm wavelength of 1.0 within 30 minutes of reaction at 37° C.

### Example 4A

### Determination of Optimal pH for Activity of Nucleolytic PPR Nuclease.

In order to determine optimal pH for enzyme nucleolytic activity reaction is conducted as described in Example 4. The solutions of pH 6.0-10.0 with 1 U enzyme in reaction mixtures containing 0, 250, 500 mM NaCl, respectively. PPR nuclease exhibits highest nucleolytic activity in a buffer of pH 8.0 at 500 mM NaCl concentration, as shown in FIG. 2.

### Example 4B

### Determination of Optimal Temperature for PPR Nuclease Nucleolytic Activity.

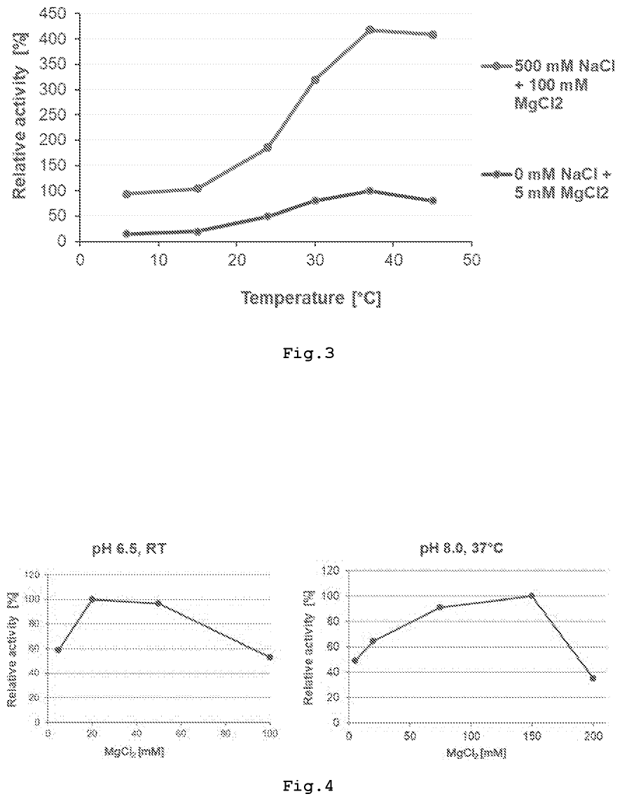

The determination of the optimal temperature for enzyme nucleolytic activity reaction is performed as described in Example 4 at various temperatures from 6° C. to 45° C. with 1 U enzyme in the reaction mixture containing 50 mM Tris, pH 8.0 and variable concentrations of MgCh (5 mM and 100 mM). The PPR nuclease maintains nucleolytic activity in the whole range of the tested temperatures (FIG. 3). However, the highest activity is exhibited at 37° C. and with high concentration of NaCl (500 mM) and MgCh (100 mM). It needs to be highlighted that in these conditions 100% of standard activity may be obtained in refrigerated conditions at 6° C., by applying the relevant salt concentration: NaCl (500 mM) and MgCh (100 mM).

### Example 4C

### Determination of Optimal Concentration of Mg²⁺ Ions for PPR Nuclease Nucleolytic Activity.

To determine the effects of Mg²⁺ ion concentration on PPR nucleolytic activity reaction is performed as described in Example 4 in solutions of varied content of Mg²⁺ ions; 5 mM to 200 mM with 1 U enzyme in the reaction mixture. In a slightly alkaline environment (pH 8.0) the PPR nuclease shows the highest nucleolytic activity in the buffer containing Mg²⁺ ions at 150 mM concentration (optimum 50-150 mM), as shown in FIG. 4. In a low-pH environment (pH 6.5) PPR shows the highest nucleolytic activity in the buffer containing Mg²⁺ ions at 20 mM concentration (optimum 20-50 mM). In all observed ranges of MgCh (5-200 mM) PPR concentrations show high specific activity.

### Example 4D

### Confirmation of Potential Inhibitor Effects on PPR Enzymatic Activity.

To determine the scope of enzyme tolerance to popular ion components used in the preparation of recombinant proteins, present in the reaction buffer, a nucleolytic reaction is performed as described in Example 4 in solutions of various amounts of individual potential inhibitors: NaCl, urea, ammonium sulphate, imidazole. The PPR nuclease retains high nucleolytic activity in the presence of increased concentrations of tested substances, as shown in Table 1.

TABLE 1
Effects of inhibitors on PPR nucleolytic activity

| Substance | Non-inhibiting concentration |
|---|---|
| NaCl/KCl | 0-1400 mM |
| Urea | 0-6000 mM |
| Ammonium sulphate | 0-200 mM |
| Imidazole | 0-400 mM |

### Example 4E

### Thermal Inactivation of PPR Enzymatic Activity

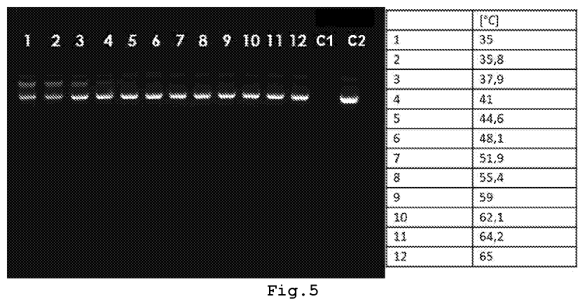

To determine the parameters of PPR nuclease inactivation series of 0.2 ml test probes for PCR are prepared containing 100 U PPR in a 50 m [symbol] reaction buffer containing 5 mM DTT, as described in Example 4A, excluding pUCIO plasmid DNA. Afterwards, the probes are incubated for 15 minutes at relevant temperatures and then for 5 minutes on ice. As a control, 100 U PPR nucleases in the same buffer are kept on ice. Following that, nucleolytic reaction is performed using a 5 m [symbol] PPR solution from the previous stage, after inactivation described in Example 4A. As a control, only plasmid DNA in the reaction buffer (negative control) and plasmid DNA with 100 U PPR kept on ice (positive control) are incubated in the same conditions as reaction trials. The degree of plasmid DNA degradation in the trials is analyzed on an agarose gel. As shown in FIG. 5, PPR is completely inactivated in the presence of DDT at 52° C. or higher.

### Example 4F

### Determination of Nucleolytic Activity of PPR, HL-SAN, Benzonase® Nucleases in Buffers of Varied Concentration of Salts and pH.

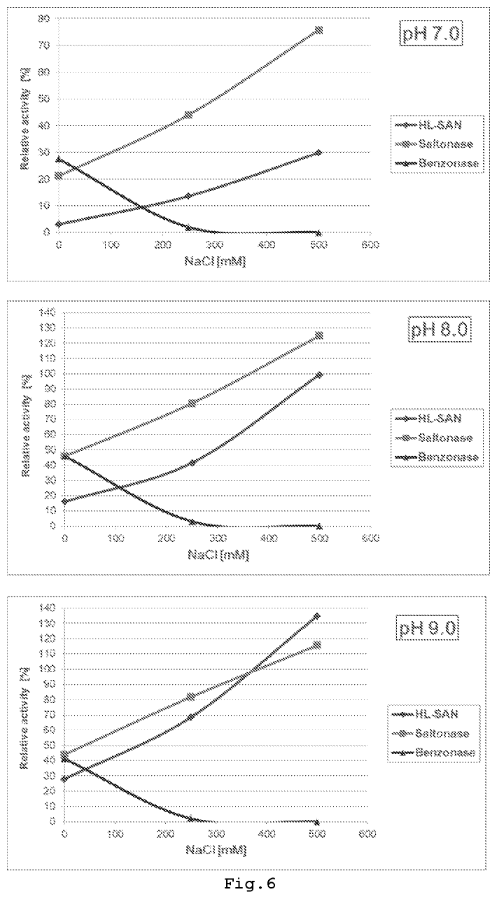

Determination of nucleolytic activity of PPR, HL-SAN and Benzonase® nucleases is performed at various temperatures; 6° C., 22° C. and 37° C., with varied addition of NaCl to the final concentration: 0, 250, 500 mM, in pH 7.0, 8.0, 9.0, respectively in the presence of 50 mM Tris, 20 mM MgCh (5 mM MgCl2 for Benzonase®). As shown in FIG. 6, the PPR nuclease exhibits the highest nucleolytic activity of all tested nucleases in the buffers with increased content of salt (250 and 500 mM NaCQ) in pH 7.0, 8.0 and 9.0 (only HL-SAN shows slightly higher activity in 500 mM NaCl and pH 9.0). The Benzonase® nuclease practically does not function in any conditions of increased salt concentration (250, 500 mM NaCl), irrespective of pH value. In conditions of lower pH (7.0, 8.0) and ambient temperature (22° C.) the PPR nuclease is significantly more active compared with HL-SAN and Benzonase®. A similar correlation was observed for 6° C. and 37° C. (data not shown).

### Example 4G

### Determination of Nucleolytic Activity of PPR, HL-SAN, Benzonase® Nucleases in DMEM Medium.

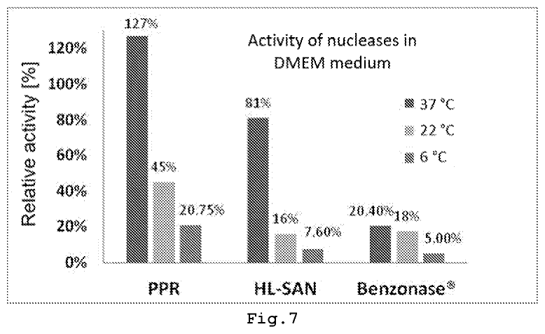

Determination of the nucleolytic activity of PPR, HL-SAN and Benzonase® nucleases are performed as described in Example 4 at various temperatures: 6° C., 22° C. and 37° C. but instead of a reaction buffer DMEM medium (commonly used in cultures of mammalian cells, e.g. CHO, HEK, for the production of recombinant proteins and virus vectors) is used. As shown in FIG. 7, the PPR nuclease, added directly to the DMEM medium at all tested temperatures shows the highest nucleolytic activity of all tested enzymes. At 37° C. PPR is six times more active than the Benzonase® nuclease. In refrigerated conditions (6° C.) and ambient temperature (22° C.) PRR is approx three times more active than the other tested nucleases, i.e. HL-SAN and Benzonase® (FIG. 7). The activity for every enzyme in conditions recommended by the manufacturer was assumed as 100% activity.

### Example 4H

### Determination of Nucleolytic Activity of PPR, HL-SAN, Benzonase® Nucleases in PBS and TBS Buffers.

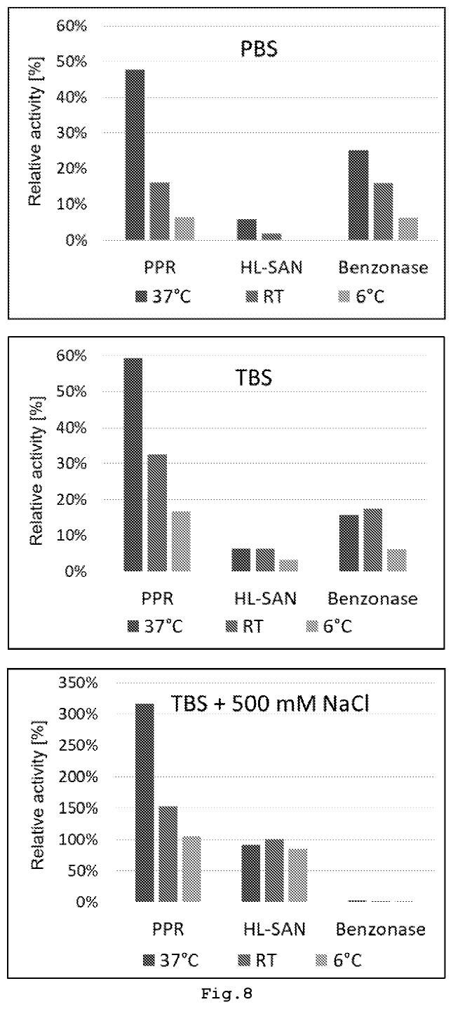

Determination of the nucleolytic activity of PPR, HL-SAN and Benzonase® nucleases is performed as described in Example 4 at 6° C., 22° C. and 37° C. but instead of a reaction buffer the following buffers are used: PBS (Phosphate Buffered Saline) pH 7.4 (10 mM Na₂HPO₄, 1.8 mM KH₂PO₄; 2.7 mM KCl; 137 mM NaCl) and TBS (Tris Buffered Saline) (50 mM Tris-Cl, pH 7.6; 150 mM NaCl), commonly used in processes of recombinant protein purification. Moreover, the activity of nucleases in TBS with the addition of 500 mM NaCl was compared. As presented in FIG. 8, the PPR nuclease in all tested buffers, i.e. PBS, TBS and TBS with high content of salt (500 mM NaCl) and at all tested temperatures shows the highest nucleolytic activity of all tested enzymes. The activity for every enzyme in conditions recommended by the manufacturer was assumed as 100% activity (FIG. 8).

### Example 5

### Application of the Recombinant PPR Nuclease

### Example 5A

### Application of the PPR Nuclease in the Production of Recombinant UDGase of Lower Host DNA Content.

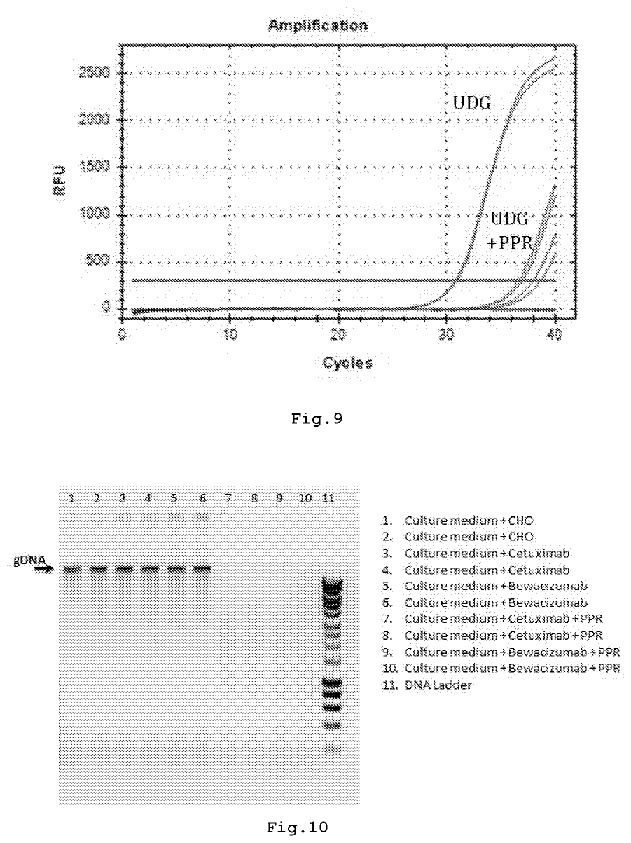

Following Example 3, the obtained PPR nuclease is used in the process of purification of other recombinant enzymes commonly used in scientific studies and molecular diagnostics, particularly in polymerases, ligases and UDGases containing significantly lower host DNA contaminants. The standard protocol of UDGase purification of E. coli bacteria has been modified so that PPR nuclease is added to the prepared bacteria lysate containing overproduced UDGase in the following manner: 40 U per 1 ml of lysate and subsequent incubation at 20-25° C. for 1 hour using a magnetic mixer set at 200 rotations/min. Consequently, the lysate is processed in accordance with standard procedures for UDGase. Content measurement of host DNA contamination is performed using the qPCR method with 16S bacteria-specific primers. UDGase purified using the additional step with the PPR nuclease contains 100 times fewer host DNA contaminants in comparison with an enzyme purified without the PPR nuclease, which is shown in FIG. 9. The UDGase enzyme purified in such a manner used in scientific studies or molecular diagnostics increases the sensitivity of the method and significantly lowers the risk of potential false positive results.

### Example 5B

### Application of the PPR Nuclease for the Removal of DNA Contamination in the Process of Monoclonal Antibody Purification of Mammalian Cells.

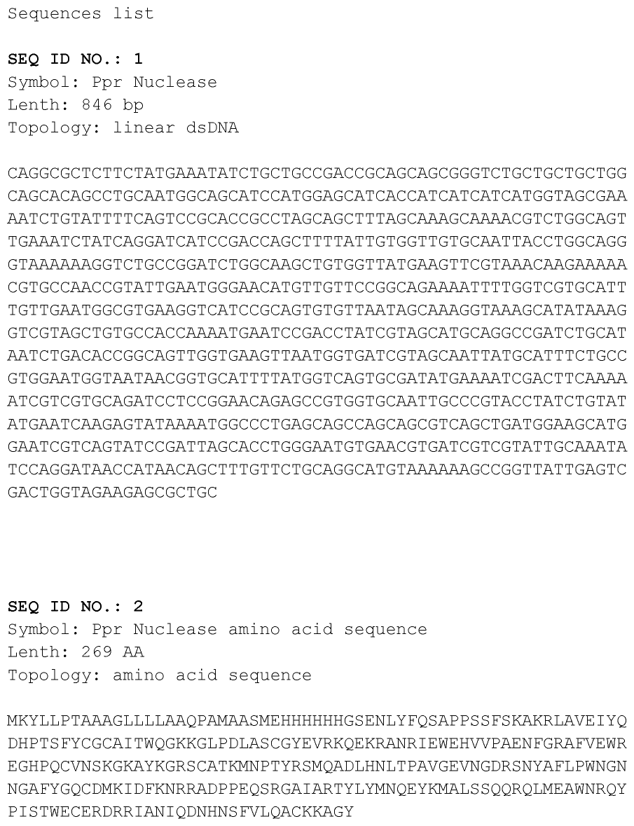

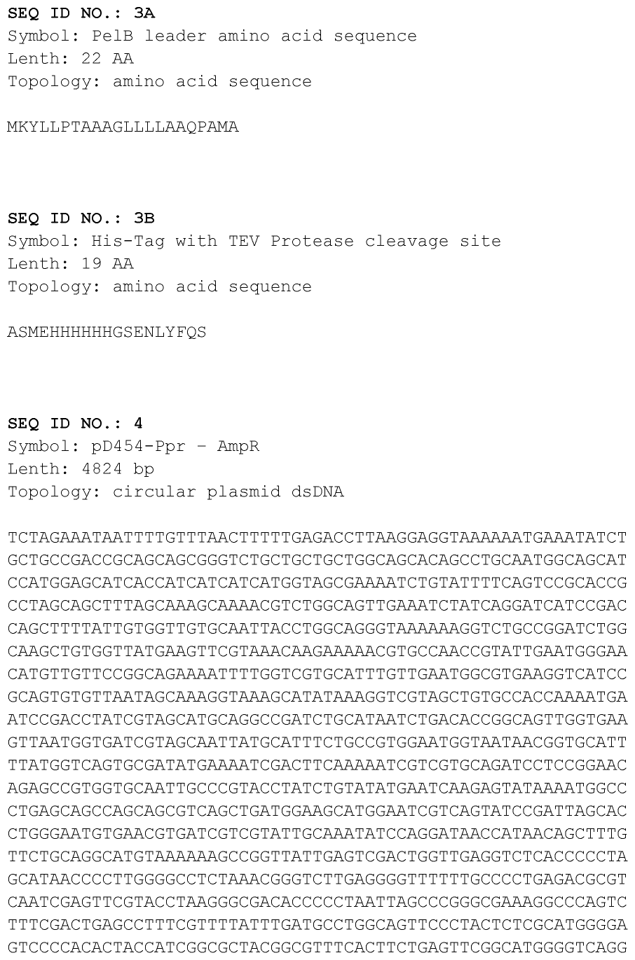

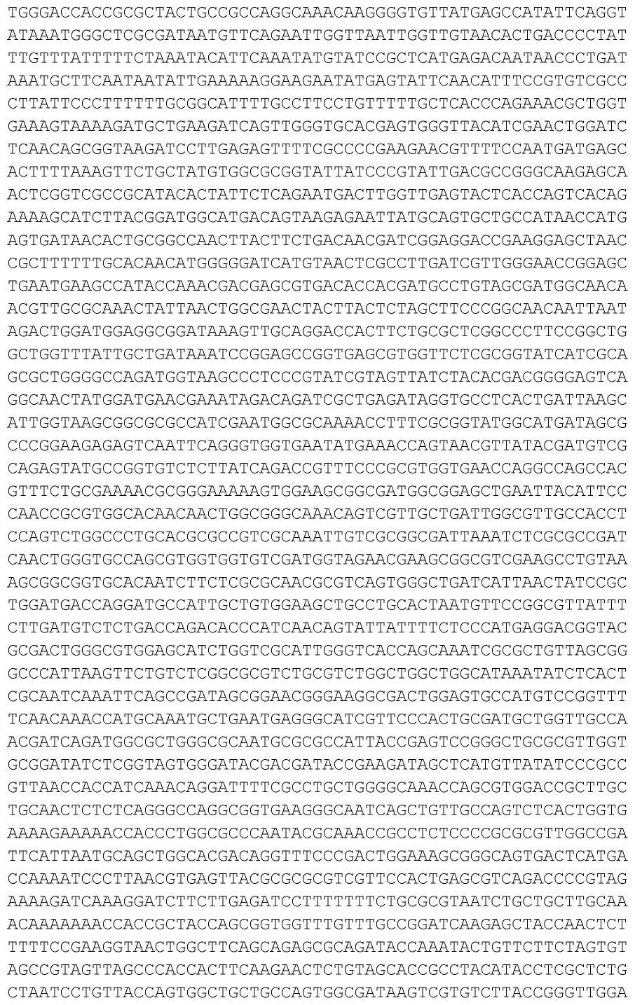

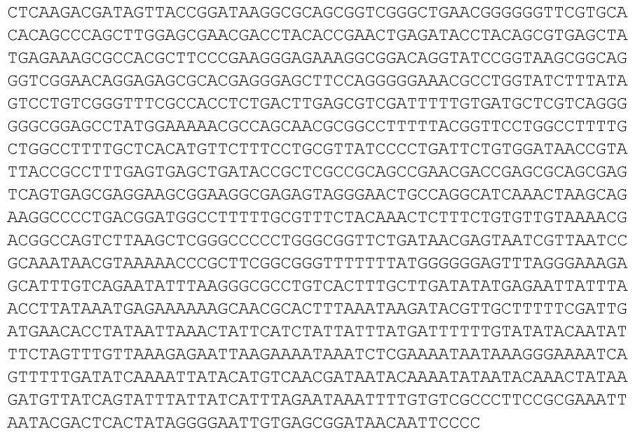

The PPR nuclease enzyme obtained as described in Example 3 is used for the removal of DNA contaminants in the process of purification of recombinant monoclonal antibodies isolated from Chinese hamster ovary (CHO) cells. Mammalian cells are cultured on a relevant medium for 5 days. Afterwards, the cells are separated by centrifugation and a supernatant is used to purify antibodies by standard chromatographic methods. In the medium, apart from antibodies and medium components, a large amount of genomic DNA derived from host cells degraded during culture is present. Performance of the preliminary stage of incubation of the post-culture medium with the PPR nuclease significantly reduces the content of DNA contamination in the medium, and thus the efficiency of antibodies binding to the stationary phase is increased. The stage of post-culture medium incubation with the addition of the PPR nuclease to 50 U/ml of medium for 60 minutes at 20-22° C. was introduced into the standard process of the purification of antibodies. After this time, from 1 ml of medium, treated and untreated with the PPR nuclease, DNA is isolated using the genomic DNA isolation kit. The total obtained DNA is loaded onto a 1% agarose gel with the addition of ethidium bromide to visualize nucleic acids. Then, the medium undergoes the standard procedure of purification of antibodies. As a control, a post-culture medium is incubated in the same conditions without the addition of the PPR nuclease. As shown in FIG. 10, the addition of the PPR nuclease significantly decreases the contamination of genomic DNA in the post-culture medium, accumulated during the growth of cells secreting cetuximab and bevacizumab antibodies outside into the medium.

1. An isolated recombinant protein comprising a PPR nuclease or an enzymatically active fragment thereof, wherein the isolated recombinant protein sequence comprises amino acids 42 to 269 of SEQ ID NO: 2 and a hexa-HIS purification tag (SEQ ID NO: 3B).

2. The isolated recombinant protein comprising PPR nuclease or its enzymatically active fragment thereof according to claim 1, wherein the recombinant protein is irreversibly inactivated after incubation comprising:
a) 15 minutes at 52° C. in the presence of 1-5 mM DTT or
b) at a temperature of 52° C. or lower for longer than 15 minutes in the presence of 1-5 mM DTT.

3. The isolated recombinant protein comprising PPR nuclease or its enzymatically active fragment thereof according to claim 1, wherein the nuclease is enzymatically active in salt concentrations of the following ranges: NaCl: 0-1400 mM, MgCl 5-200 mM, urea: 0-6000 mM, ammonium sulfate: 0-200 mM, imidazole: 0-400 mM.

4. (canceled)

5. The isolated recombinant protein comprising PPR nuclease or its enzymatically active fragment thereof according to claim 1, wherein the recombinant protein is expressed from a nucleic acid sequence according to SEQ ID NO: 1 or fragment thereof.

6. An expression plasmid pD454-PPR-AmpR (SEQ ID NO: 4), comprising a nucleic acid sequence encoding the isolated recombinant protein comprising PPR nuclease or its enzymatically active fragment thereof of claim 1 wherein the nucleic acid sequence further comprises a T7 phage promoter or another promoter active in an E. coli expression system.

7. A recombinant Escherichia coli strain of AmpR transformed by the expression plasmid of claim 6.

8. A method of producing a PPR nuclease protein according to claim 1, the method comprising culturing the recombinant strain of Escherichia of claim 7 comprising an expression vector pD454-PPR-AmpR on a medium;
inducing PPR nuclease gene expression by adding IPTG- and,
isolating and purifying the PPR nuclease protein.

9. A method of isolation and purification of the PPR nuclease, or its enzymatically active fragment as defined in claim 1, comprising expression of the PPR nuclease or its fragment in a host cell, and followed by separation of the PPR nuclease from the host cell and/or cell culture medium thereof.

10. A method of using the isolated recombinant protein comprising PPR nuclease or its enzymatically active fragment thereof according to claim 1, wherein the use is selected from
i) purification of recombinant proteins;
ii) purification of recombinant proteins to reduce DNA content;
iii) decontamination of reagents and reaction mixtures for PCR, qPCR, RT-PCR, RT-qPCR, RCA, LAMP and NGS;
iv) decontamination of reagents and reaction mixtures for PCR, qPCR, RT-PCR, RT-qPCR, RCA, LAMP and NGS to increase sensitivity and specificity of the relevant genetic analyses;
v) virus vector purification;
vi) purification of exosomes;
vii) purification of pharmaceutical grade proteins, enzymes, antibodies, or vaccination antigens;
viii) purification of veterinary grade proteins, enzymes, antibodies, or vaccination antigens; or
ix) purification of cosmetic grade proteins, enzymes, or antigens;
x) removal of nucleic acid contamination in culture media for mammalian fermentation and microbiological processes.

11. The method according to claim 10, wherein the virus vector is selected from lentiviruses [LV], adenoviruses [AV, AAV] and retroviruses [RV].

12. (canceled)

13. (canceled)

14. (canceled)

15. (canceled)

16. The method of claim 8, wherein the expression vector comprises SEQ ID NO: 1.

17. An isolated recombinant protein comprising a PPR nuclease or an enzymatically-active fragment thereof, wherein the isolated recombinant protein comprises amino acids 23 to 269 of SEQ ID NO: 2.

18. A composition comprising an isolated recombinant protein comprising a PPR nuclease or enzymatically-active fragment thereof according to claim 1 comprising salts at a concentration selected from one or more of the following concentrations: NaCl: 0-1400 mM, MgCl 5-200 mM, urea: 0-6000 mM, ammonium sulfate: 0-200 mM, and imidazole: 0-400 mM.

19. The method according to claim 10, wherein the virus vector purification is used in gene and cell therapies or chimeric antigen receptor [CAR] T-cell immunotherapy.


SEQUENCE LISTING (5 sequences)

SEQ ID NO: 1 - DNA, 846 bp, Artificial Sequence
  Synthetic DNA
caggcgctcttctatgaaatatctgctgccgaccgcagcagcgggtctgctgctgctggcagcacagcctgcaatggcagcatccatggagcatcaccatcatcatcatggtagcgaaaatctgtattttcagtccgcaccgcctagcagctttagcaaagcaaaacgtctggcagttgaaatctatcaggatcatccgaccagcttttattgtggttgtgcaattacctggcagggtaaaaaaggtctgccggatctggcaagctgtggttatgaagttcgtaaacaagaaaaacgtgccaaccgtattgaatgggaacatgttgttccggcagaaaattttggtcgtgcatttgttgaatggcgtgaaggtcatccgcagtgtgttaatagcaaaggtaaagcatataaaggtcgtagctgtgccaccaaaatgaatccgacctatcgtagcatgcaggccgatctgcataatctgacaccggcagttggtgaagttaatggtgatcgtagcaattatgcatttctgccgtggaatggtaataacggtgcattttatggtcagtgcgatatgaaaatcgacttcaaaaatcgtcgtgcagatcctccggaacagagccgtggtgcaattgcccgtacctatctgtatatgaatcaagagtataaaatggccctgagcagccagcagcgtcagctgatggaagcatggaatcgtcagtatccgattagcacctgggaatgtgaacgtgatcgtcgtattgcaaatatccaggataaccataacagctttgttctgcaggcatgtaaaaaagccggttattgagtcgactggtagaagagcgctgc

SEQ ID NO: 2 - protein, 269 aa, Artificial Sequence
  Synthetic Peptide
MKYLLPTAAAGLLLLAAQPAMAASMEHHHHHHGSENLYFQSAPPSSFSKAKRLAVEIYQDHPTSFYCGCAITWQGKKGLPDLASCGYEVRKQEKRANRIEWEHVVPAENFGRAFVEWREGHPQCVNSKGKAYKGRSCATKMNPTYRSMQADLHNLTPAVGEVNGDRSNYAFLPWNGNNGAFYGQCDMKIDFKNRRADPPEQSRGAIARTYLYMNQEYKMALSSQQRQLMEAWNRQYPISTWECERDRRIANIQDNHNSFVLQACKKAGY

SEQ ID NO: 3 - protein, 22 aa, Artificial Sequence
  Synthetic Peptide
MKYLLPTAAAGLLLLAAQPAMA

SEQ ID NO: 4 - protein, 19 aa, Artificial Sequence
  Synthetic Peptide
ASMEHHHHHHGSENLYFQS

SEQ ID NO: 5 - DNA, 4824 bp, Artificial Sequence
  Synthetic DNA
tctagaaataattttgtttaactttttgagaccttaaggaggtaaaaaatgaaatatctgctgccgaccgcagcagcgggtctgctgctgctggcagcacagcctgcaatggcagcatccatggagcatcaccatcatcatcatggtagcgaaaatctgtattttcagtccgcaccgcctagcagctttagcaaagcaaaacgtctggcagttgaaatctatcaggatcatccgaccagcttttattgtggttgtgcaattacctggcagggtaaaaaaggtctgccggatctggcaagctgtggttatgaagttcgtaaacaagaaaaacgtgccaaccgtattgaatgggaacatgttgttccggcagaaaattttggtcgtgcatttgttgaatggcgtgaaggtcatccgcagtgtgttaatagcaaaggtaaagcatataaaggtcgtagctgtgccaccaaaatgaatccgacctatcgtagcatgcaggccgatctgcataatctgacaccggcagttggtgaagttaatggtgatcgtagcaattatgcatttctgccgtggaatggtaataacggtgcattttatggtcagtgcgatatgaaaatcgacttcaaaaatcgtcgtgcagatcctccggaacagagccgtggtgcaattgcccgtacctatctgtatatgaatcaagagtataaaatggccctgagcagccagcagcgtcagctgatggaagcatggaatcgtcagtatccgattagcacctgggaatgtgaacgtgatcgtcgtattgcaaatatccaggataaccataacagctttgttctgcaggcatgtaaaaaagccggttattgagtcgactggttgaggtctcaccccctagcataaccccttggggcctctaaacgggtcttgaggggttttttgcccctgagacgcgtcaatcgagttcgtacctaagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaagcggcgcgccatcgaatggcgcaaaacctttcgcggtatggcatgatagcgcccggaagagagtcaattcagggtggtgaatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtgactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttccgcgaaattaatacgactcactataggggaattgtgagcggataacaattcccc